(19) United States
(12) Patent Application Publication
(10) Pub. No.: US 2017/0143219 A1
(43) Pub. Date: May 25, 2017

(54) Emergency Medical Alert Wearable

(71) Applicant: Melissa Ciecko, Holland Patent, NY (US)

(72) Inventor: Melissa Ciecko, Holland Patent, NY (US)

(21) Appl. No.: 15/357,718

(22) Filed: Nov. 21, 2016

Related U.S. Application Data
(60) Provisional application No. 62/257,377, filed on Nov. 19, 2015.

(51) Int. Cl. A61B 5/024; A61B 5/11; A61B 5/00; A61B 5/021

(52) U.S. Cl. CPC A61B 5/02438; A61B 5/021; A61B 5/1112; A61B 5/0022; A61B 5/002; A61B 5/7405; A61B 5/7282; A61B 5/681; A61B 5/742

(57) ABSTRACT

An emergency medical alert wearable. The wearable includes sensors that are configured to monitor various physiological parameters of the wearer, including blood pressure and heart rate. When the wearable detects a deviation in the monitored physiological parameters, the wearable emits an audible alert and transmits a notification, which includes the location of the wearable, to an emergency medical service. The wearable thereby notifies both any individual who may be in the vicinity of the wearer and the appropriate authorities that the wearer is potentially suffering from an emergency medical condition and requires assistance.

### CROSS REFERENCE TO RELATED APPLICATIONS

This application claims the benefit of U.S. Provisional Application No. 62/257377 filed on Nov. 19, 2015. The above identified patent application is herein incorporated by reference in its entirety to provide continuity of disclosure.

### BACKGROUND OF THE INVENTION

If an individual is alone when they suffer from certain types of emergency medical conditions, e.g., heart attacks, potentially preventable injuries can become much more serious and potentially even lead to death. This is particularly true for emergency medical conditions that can render the individual unconscious because then the individual is completely unable to call for help or otherwise access a telephone. Wearable technology that utilizes sensors to monitor various physiological conditions of the user can be harnessed in order to assist in negating these preventable injuries and deaths. If a wearable device automatically alerted any individuals in the vicinity of the wearer and contacted the appropriate medical authorities in the event of an emergency medical condition, then the wearer of such a device could receive medical assistance regardless of whether the user is rendered unconscious or otherwise unable to seek assistance.

### SUMMARY OF THE INVENTION

In view of the foregoing disadvantages inherent in the known types of medical alert wearables now present in the prior art, the present invention provides a wearable comprising: a first sensor configured to detect a heart rate of a wearer; a second sensor configured to detect a blood pressure of the wearer; a display configured to depict a visual representation of the heart rate and the blood pressure of the wearer; a GPS receiver configured to receive a position of the wearable; a transceiver configured to transmit an alert to an emergency medical service, the alert including the position of the wearable; a speaker configured to emit an audible alert; a logic that is at least partially stored in a non-transitory computer readable medium and that, when executed at least in part by a processor, causes the wearable to perform a method, the method comprising: transmitting the alert to the emergency medical service if at least one of the heart rate and the blood pressure of the wearer fall outside a tolerance range; emitting the audible alert via the speaker if at least one of the heart rate and the blood pressure of the wearer fall outside the tolerance range.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of a medical alert wearable.

FIG. 2 shows a perspective view of a medical alert wearable.

FIG. 3 shows a diagram of a medical alert wearable sending an alert to an emergency medical service.

FIG. 4 shows a functional block diagram f a medical alert wearable.

### DETAILED DESCRIPTION OF THE INVENTION

Reference is made herein to the attached drawings. Like reference numerals are used throughout the drawings to depict like or similar elements of the medical alert bracelet. The figures are intended for representative purposes only and should not be considered to be limiting in any respect.

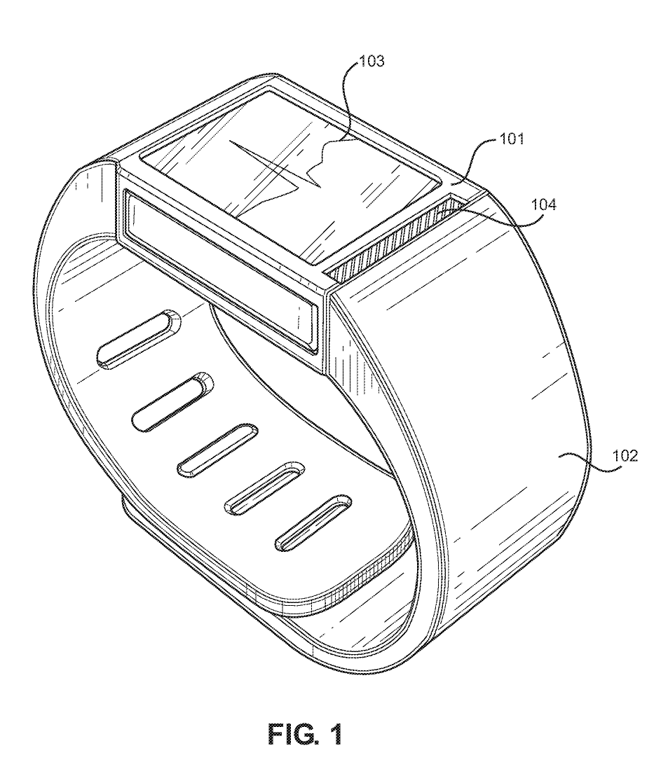

Referring now to FIG. 1, there is shown a perspective view of a medical alert wearable. The present invention is directed generally to a wearable, i.e., an article of clothing or accessory incorporating computers, electronics, and/or sensors. The wearable is configured to monitor physiological conditions of the user via sensors. If the wearable detects that the monitored physiological conditions fall outside a tolerance range, then the wearable emits an audible alert and contacts local emergency medical services. In one embodiment, the medical alert wearable includes a housing 101 supporting a display 103 and a variety of embedded electronics. The display 103 is configured to display, e.g., a heart rate and a blood pressure of the wearer as sensed by one or more sensors of the wearable. This heart rate and blood pressure information depicted by the display 103 can be represented numerically, as a graph, or via any other representation capable of relaying the data to a user. The display 103 allows a wearer to continually visualize the parameters tracked by the wearable

The wearable further includes a speaker 104 disposed on the housing 101. The speaker 104 is configured to emit an audible alert if an emergency condition is detected by the sensors of the wearable. The audible alert is intended to alert the wearer and individuals around the wearer that the wearer is potentially suffering from or about to suffer from an emergency medical condition or is falling asleep. An emergency condition is defined as when a parameter or combination of parameters tracked by the sensors meet a pre-programmed criterion as determined by the logic of the wearable. For example, an emergency condition could include the wearer's heart rate falling below a threshold level. When a criterion is determined to be satisfied via data received from the sensors as monitored by the logic, the logic causes the speaker 104 to emit an audible alert, among other actions.

The wearable can take a variety of forms. In the depicted embodiment, the wearable is generally a bracelet including a band 102 securable about, e.g., a wrist of a user. However, the wearable can take any number of forms such as are known in the art.

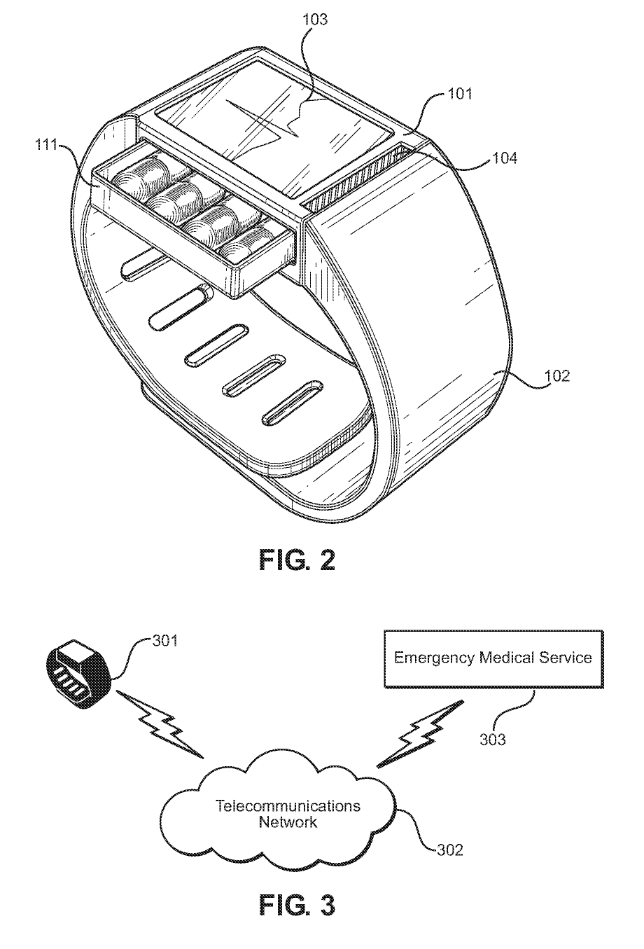

Referring now to FIG. 2, there is shown a perspective view of a medical alert wearable. In one embodiment, the wearable further includes a compartment 111 that is extendable from the housing 101. The compartment can be utilized, e.g., for storing medication usable in the event of a medical emergency. In one embodiment of the compartment 111, the compartment 111 is sized to snugly receive pills therein. In one such embodiment, the compartment 111 has a width substantially equal to a length of a pill and a length substantially equal to the combined widths of a plurality of pills. This allows the pills to be stored within the compartment 111 in an organized manner.

Referring now to FIG. 3, there is shown a diagram of a medical alert wearable sending an alert to an emergency medical service. The wearable 301 includes a transceiver that transmits a notification to an emergency medical service 303 when the wearable 301 detects that the monitored physiological conditions of the wearer fall outside a tolerance range. In an illustrative embodiment, the transceiver of the wearable 301 transmits the notification via a telecommunications network, e.g., the Internet or a mobile telephonic network. The emergency medical services 303 with which the wearable 301 is configured to communicate include, e.g., contacting 944 or another such service configured to contact and dispatch emergency responders.

The notification transmitted by the wearable 301 to an emergency medical service 303 occurs automatically upon the wearable 301 detecting the appropriate physiological conditions of the wearer. The notification transmitted to the emergency medical service 303 can include a text message, an automated voice message, or any other such notification. In one embodiment, the notification includes the current location of the wearable 301 in order to assist emergency personnel in responding to the potential medical emergency.

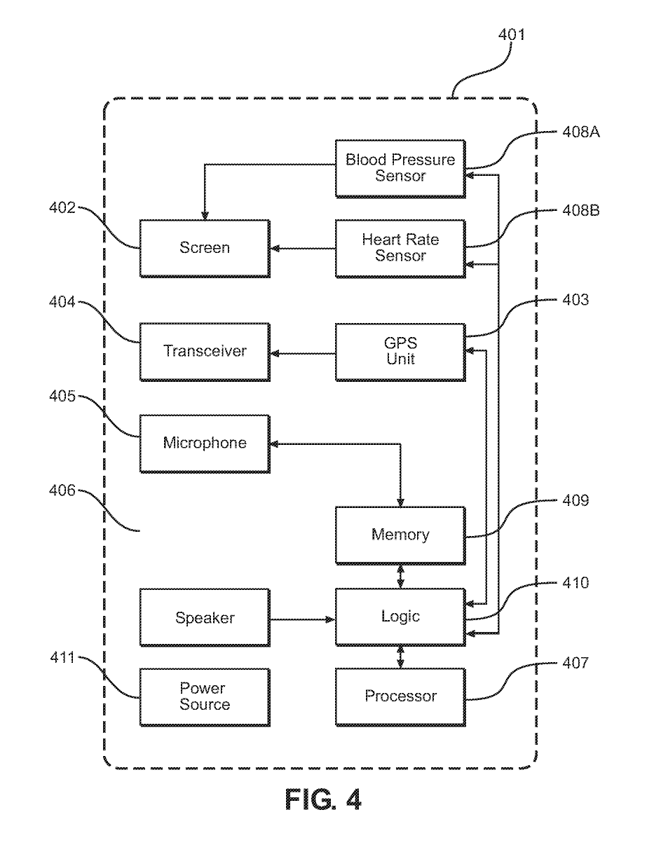

Referring now to FIG. 4, there is shown a functional block diagram of a medical alert wearable. The wearable 401 includes at least a pair of sensors that are configured to monitor various physiological parameters of the wearer. The sensors include first sensor 408A that configured to detect the blood pressure of the wearer and a second sensor 408B that is configured to detect the heart rate of the wearer. Both of the sensors 401A, 408B are operably connected to the display or screen 402, which is configured to textually or graphically display the values of the monitored parameters thereon. The screen 402 includes any type of electronic visual display known in the art, e.g., an LCD display.

The wearable 401 further includes a transceiver 401 that is configured to communicate a notification to an emergency medical service, e.g., via a telecommunications network or another such network. The notification relayed by the transceiver 401 can be textual, graphical, audible, or any other known medium for communicating a message. A GPS unit 403, i.e., a GPS receiver, is operably connected to the transceiver 401. The GPS unit 403 is configured to relay the position of the wearable 401 to the transceiver 404, which then in turn transmits the position of the wearable 401 to the emergency medical service, along with the notification, when triggered by the logic 410.

The wearable 401 further includes a logic 410, which is operably connected to the sensors 408A, 408B and the assembly of the GPS unit 403 and the transmitter 404. As used herein, “logic” refers to (i) logic implemented as computer instructions and/or data within one or more computer processes and/or (ii) logic implemented in electronic circuitry. In one embodiment, the logic is stored at least partially on a non-transitory computer readable medium, i.e., a memory 409, and is operably connected to a processor 407, which executes the logic 410.

The logic 410 monitors the blood pressure and the heart rate of the wearer via the first sensor 408A and the second sensor 408B, respectively. If either of the aforementioned physiological parameters of the wearer fall outside a tolerance range, then the logic 410 determines that the wearer is potentially suffering from an emergency medical condition and initiates the transmission of the notification and the location of the wearable 401 to the emergency medical service via the transceiver 404. In one embodiment, the tolerance range for each of the physiological parameters is a static value that is either preset by the user or by the manufacturer of the wearable 401. In another embodiment, the tolerance range for each of the physiological parameters is instead determined by the wearable 401 itself by, e.g., taking a moving average of each particular physiological parameter. In this embodiment, deviations from the moving average of a physiological parameter by a preset factor trigger the logic 410 to determine that the user is potentially suffering from an emergency medical condition.

In sum, the logic 410 continuously monitors the wearer of the wearable 401 for substantial deviations in the user's basal or resting blood pressure and heart rate. Substantial deviations in these parameters, either alone or in combination, can be used to indicate a variety of different emergency medical conditions. For example, an acute drop in blood pressure can indicate heart failure. As another example, an acute increase in heart rate can indicate atrial or supraventricular tachycardia. In any of these types of emergency medical conditions, the sufferer can potentially be rendered unconscious, preventing the individual from receiving medical assistance if alone. In such situations, the wearable 401 would automatically contact the relevant authorities with the location of the wearer so that assistance can be dispatched and emit an audible alert via the speaker 406 so that any individuals in the immediate vicinity of the wearer are notified that the wearer is undergoing a medical emergency, without any need for the wearer to be conscious and call for help.

In one embodiment, the transceiver 404 additionally includes a short-range wireless connection, e.g., a Bluetooth connection, so that the wearable 401 can be connected to an automobile or another such secondary device. In this embodiment, the wearable 401 can emit additional alerts, without necessarily automatically contacting an emergency medical service. For example, if the logic 410 determines that the heart rate of the wearer is dropping via the second sensor 408B, but is not dropping dangerously, that can nonetheless indicate that the wearer is falling asleep. Therefore, if the wearable 401 is connected to the vehicle and the logic 410 determines that the wearer is potentially falling asleep, then the wearable 401 can emit an audible alert via the speaker 406, without transmitting a notification to an emergency medical service, in order to wake the wearer up.

In one embodiment, the wearable 401 further includes a microphone 405 that is operably connected with the memory 409. When activated, the microphone 405 can be used by the wearer to record any sounds or messages deemed necessary by the wearer. For example, the wearer can utilize the microphone 405 to record notes on the medication that the user needs to take. In one embodiment, the wearer can utilize the microphone 405 to record a vocal message that serves as the notification that is relayed to the emergency medical service via the transmitter 404. In this embodiment, when an emergency medical condition is detected by the logic 410, then the logic 410 retrieve the pre-recorded message from the memory 409 and then transmits the pre-recorded message via the transceiver 404.

The wearable 401 can incorporate various other electronics, including a power source 411 that is operably connected to the various other electronic components of the device such that they can be powered thereby.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A wearable, comprising:
a first sensor configured to detect a heart rate of a wearer;
a second sensor configured to detect a blood pressure of the wearer;
a display configured to depict a visual representation of the heart rate and the blood pressure of the wearer;
a GPS receiver configured to receive a position of the wearable;
a transceiver configured to transmit an alert to an emergency medical service, the alert including the position of the wearable;
a speaker configured to emit an audible alert;
a logic that is at least partially stored in a non-transitory computer readable medium and that, when executed at least in part by a processor, causes the wearable to perform a method, the method comprising:
transmitting the alert to the emergency medical service if at least one of the heart rate and the blood pressure of the wearer fall outside a tolerance range;
emitting the audible alert via the speaker if at least one of the heart rate and the blood pressure of the wearer fall outside the tolerance range.

2) The wearable of claim 1, further comprising a compartment extendable from the wearable.

3) The wearable of claim 1, further comprising a microphone, wherein the microphone is operably connected to the non-transitory computer readable medium and configured to store recorded sound thereon.

4) The wearable of claim 1, wherein the wearable is a bracelet including a band.

5) The wearable of claim 1, wherein the transceiver transmits the alert via a telecommunication network.

6) The wearable of claim 1, wherein the transceiver includes a Bluetooth connection, the Bluetooth connection configured to synch the wearable with a vehicle.

7) The wearable of claim 1, wherein the tolerance range is preset.

8) The wearable of claim 1, wherein the tolerance range is a moving average of each of the heart rate and the blood pressure of the user offset by a factor.
